(19) United States
(12) Patent Application Publication
(10) Pub. No.: US 2003/0208218 A1
(43) Pub. Date: Nov. 6, 2003

(54) Ultrasonic needle cover

(76) Inventors: Kenneth E. Kadziauskas, Coto de Caza, CA (US); Susanne M. Roslon, Dana Point, CA (US)

Correspondence Address: WALTER A. HACKLER, 2372 S.E. BRISTOL, SUITE B, NEWPORT BEACH, CA, 92660-0755, US

(21) Appl. No.: 10/138,545

(22) Filed: May 2, 2002

(51) Int. Cl. A61B 17/32

(52) U.S. Cl. 606/169

(57) ABSTRACT

A phacoemulsification handpiece that includes a vibrated needle for emulsification of tissue and a hub interconnecting the needle with a sonic power source. Apparatus, including a cover, is provided and sized for enveloping the hub and a proximal end of the needle for preventing wicked by the needle to the hub, from being outwardly sprayed by the hub during surgical operation.

The present invention generally relates to apparatus and methods for removing cataractous lens tissue. More specifically, the present invention is directed to apparatus for cataract extraction utilizing a sonic or ultrasonic handpiece vibrating a bare needle with a separate irrigation source otherwise known as bi-manual cataract extraction.

The natural crystalline lens of a human eye may become cloudy and eventually opaque through natural aging or otherwise through trauma or disease. This condition is known as a cataract.

A well known phacoemulsification procedure involves placing a sleeved needle through a cornea incision made through the limbus or cornea. The needle is ultrasonically vibrated in order that a tip portion thereof may be utilized for emulsification of the cataractous lens.

The needle and this procedure typically utilizes a lumen therethrough for aspiration of the emulsified tissue and an annulus established between the sleeve and the needle is utilized for providing irrigation fluid through the eye.

Alternatively a bimanual phacoemulsification system may be utilized in which an unsleeved needle is utilized for emulsification of lens tissue. See U.S. patent application Ser. No. 09/894,503 filed Jun. 28, 2001. This application is to be incorporated in its entity by this specific reference thereto. A lumen through the needle provides for aspiration of the emulsified tissue and in this procedure a separate needle, or conduit, inserted into the eye provides for the necessary irrigation fluid.

The fluid, which is saline, prevents the cornea from collapsing as the lens material is emulsified and aspirated and further promotes the aspiration of emulsified cataractous tissue from the eye.

The use of an unsleeved aspiration/emulsification needle enables a smaller incision through the limbus or cornea. However an absolute seal around the needle entering the incision, or wound, is not obtainable. Accordingly some leakage occurs between the needle and the incision. Because the needle is rigid and nonconforming there is never a perfect match to the shape or contour of the eye in the incision. This results in small gaps which are not normally sealed because the collagen fiber structure of the cornea resists deformation and does not readily assume this shape of the needle.

Consequently there is some leakage of fluid exterior to the needle.

Since the needle is sonically or ultra sonically vibrated there is a natural wicking, or creeping of the fluid along the needle exterior.

As this fluid contacts a needle hub interconnecting the needle with the handpiece dispersment, or spray, of the fluid may occur which may interfere with the visual field of the surgeon.

The present invention overcomes this problem which, heretofore, in connection with sleeved needles, has not been recognized.

### SUMMARY OF THE INVENTION

Apparatus in accordance with the present invention for use with the phacoemulsification handpiece having a vibrated needle for emulsification of tissue and the hub interconnecting the needle with a sonic power source generally includes a cover, sized for enveloping the hub at a proximal end of the needle for preventing fluid, wicked by the needle to the hub, from being outwardly sprayed by the hub.

In combination, the present invention provides for a handpiece which includes a housing and a sonic power source disposed within the housing.

A needle is provided having a hub disposed on a proximal end thereof connected to the power source and a cover is provided for enveloping the hub and a portion of a needle proximate end, for preventing fluid, accumulating on an exterior surface of the hub, from being dispersed outwardly from the hub.

More specifically in accordance with the present invention the cover includes a proximal end fixed to the handpiece and a distal end surrounding the needle proximal end and forming an annulus thereabout. The annulus enables fluid to pass under the cover. Thus, fluid contacting the hub under the cover is prevented from being outwardly sprayed or dispersed due to the cover. Preferably the cover is formed from a resilient material.

In another embodiment of the present invention, the cover includes a proximal end which is fixed to the handpiece and a distal end surrounding and abutting the needle proximal end for preventing fluid from reaching the hub. In this embodiment any fluid migrating, or wicking, toward the hub is received by the cover. The cover, being resilient, does not vibrate in a manner dispersing or spraying the accumulated fluid. Accordingly there is no misting, or spraying into the visual field of the surgeon.

A method in accordance with the present invention for preventing dispersing, or spraying, or fluid from a hub interconnecting a phacoemulsification needle and a handpiece comprises a step of disposing a cover over the hub and a proximal end of the needle.

### BRIEF DESCRIPTION OF THE DRAWINGS

The present invention will be more clearly understood with the respect to the following detailed description and the accompanying drawings of which:

FIG. 1 shows a prior art bare needle phacoemulsification handpiece illustrating a spray or mist emanating therefrom;

FIG. 2 is an exploded perspective view of the apparatus and handpiece in accordance with the present invention;

FIG. 3 is an enlarged cross section view of the apparatus shown in FIG. 3; and

FIG. 4 is an enlarged cross-section view of an alternative embodiment of the present invention.

### DETAILED DESCRIPTION

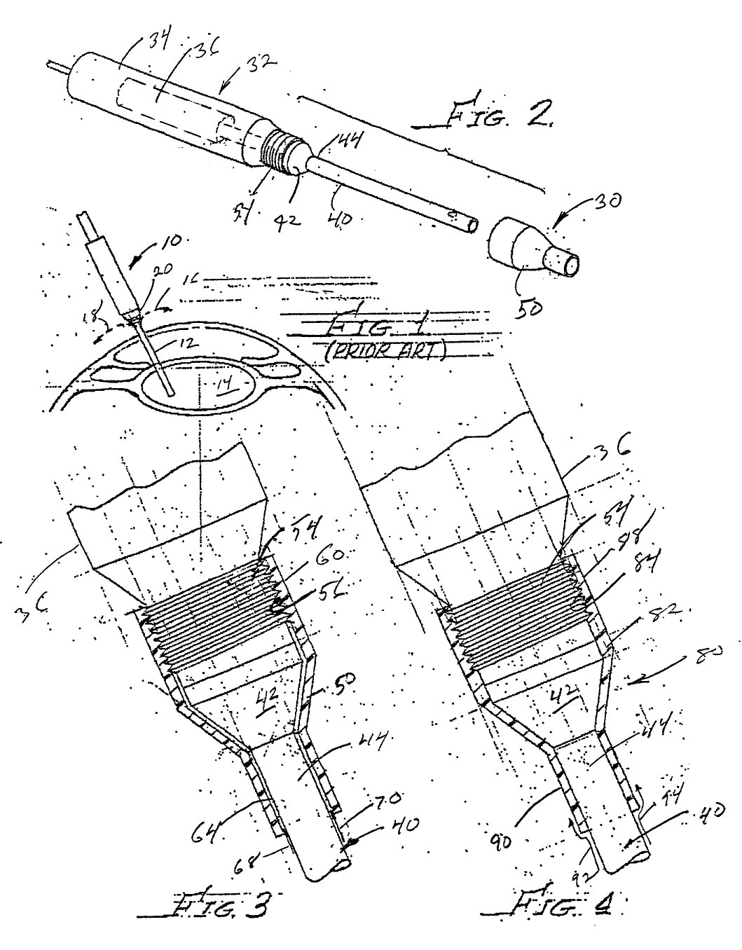

FIG. 1 illustrates a prior art phacoemulsification handpiece 10 with an unsleeved needle 12 as it may be used for phacoemulsification of a lens 14. As illustrated by arrows 16, 18 fluid wicking up the needle 12 is dispersed or sprayed from a handpiece hub 20 as indicated by the arrows 16, 18. The spray or mist may interfere with the surgeons field of vision and accordingly is undesirable.

With reference to FIG. 2, there is shown apparatus 30 for use with a phacoemulsification handpiece 32, which also forms a part of the present invention. The handpiece 32 may be made in accordance with U.S. patent application Ser. No. 09/894,503 hereinabove referenced and includes a housing 54, a sonic power source 36 with a needle 40 having a hub 42 disposed on a proximal end 44 thereof and connected to the power source 36.

As more clearly shown in FIG. 3, the apparatus 30 comprises a cover 50 sized for enveloping the hub 42 and a proximal end 44 of the needle 40. Attachment of the cover 50 may be through the use of threads 54 on the hub 42 and internal threads 56 on a distal end 60 of a cover 50.

As also shown in FIG. 3, the cover 50 surrounds the needle proximal end 44 and forms an annulus 64 thereabout. This annulus 64 enables fluid indicated by the arrows 68, 70 to pass under the cover 50.

As a result any fluid wicking or creeping along the needle 40 onto the hub 42 cannot be dispersed or sprayed by the hub 42 because of the protective cover 50.

Turning to FIG. 4, there is shown an alternative embodiment 80 in accordance with the present invention which includes a cover 82 attached by threads 84 to hub 54 threads at a distal end 88 of the cover 82. Common reference numerals in FIG. 4 representing identical or substantially similar elements, as hereinabove described in connection with the embodiment 30 shown in FIGS. 2 and 3.

As shown in FIG. 4, the cover 82 includes a distal end 90 which surrounds and abuts the needle proximal end 44 thus preventing fluid, as indicated by the arrows 92, 94, from reaching the hub 44.

The cover 82, as well as the cover 50, are preferably formed from an elastic material, such as, for example silicone. The elasticity of the silicone cover 80 prevents vibration from the hub 42 from being transferred therethrough in a manner causing dispersion, spraying or misting therefrom. The cover 82 may have a thickness of greater than about 0.05 inches, the thickness being determined by the elasticity of the elastic material. While the fluid, indicated by the arrows 92, 94, may creep along the needle 40 and over the cover 82 no spraying occurs which may interfere the surgeons field of vision.

Although there has been hereinabove described an apparatus and handpiece in accordance with the present invention for the purpose of illustrating the manner in which the invention may be used to advantage, it will be appreciated that the invention is not limited thereto. Accordingly, any and all modifications, variations, or equivalent arrangements which may occur to those skilled in the art, should be considered to be within the scope of the invention as defined in the appended claims.

What is claimed is:

1. Apparatus for use with a phacoemulsification handpiece having a vibrated needle for emulsification of tissue and a hub interconnecting the needle with a sonic power source, the apparatus comprising a cover, sized for enveloping the hub and a proximal end of the needle, for preventing fluid, wicked by the needle to said hub, from being outwardly sprayed by the hub.

2. The apparatus according to claim 1 wherein said cover includes a proximal end, fixed to the handpiece, and a distal end surrounding the needle proximal end and forming an annulus thereabout, said annulus enabling fluid to pass under the cover.

3. The apparatus according to claim 1 or 2 wherein said cover is formed from a resilient material.

4. The apparatus according to claim 3 wherein said resilient material comprises silicone.

5. The apparatus according to claim 1 wherein said cover includes a proximal end, fixed to the handpiece, and a distal end surrounding and abutting the needle proximal end for preventing fluid from reaching said hub.

6. The apparatus according to claim 5 wherein said cover is formed from a resilient material.

7. The apparatus according to claim 6 wherein said resilient material comprises silicone.

8. A handpiece for phacoemulsification of tissue comprising:
a housing;
a sonic power source disposed in said housing;
a needle having a hub disposed on a proximal end thereof connected to the power source; and
a cover, enveloping said hub and a portion of the needle proximal end, for preventing fluid, accumulating on an exterior surface of said hub, from being dispersed outwardly from said hub.

9. The handpiece according to claim 8 wherein said cover includes a proximal end, fixed to the handpiece and a distal end surrounding the needle proximate end portion and forming an annulus thereabout, said annulus enabling fluid to pass under the cover.

10. The handpiece according to claim 8 or 9 wherein said cover is formed from a resilient material.

11. The handpiece according to claim 10 wherein said resilient material comprises silicone.

12. A handpiece for phacoemulsification of tissue comprising:
a housing;
a sonic power source disposed in said housing;
a needle having a hub disposed in a proximal end thereof connected to the power source; and
a cover, enveloping said hub and a portion of the needle proximal end, for preventing fluid wicked in an exterior surface of said needle, from reaching said hub.

13. The handpiece according to claim 12 wherein said cover is formed from a resilient material.

14. The handpiece according to claim 13 wherein said resilient material comprising silicone.

15. A method for preventing dispersing of fluid from a hub interconnecting a phacoemulsification needle and a handpiece, said method comprising the step of disposing a cover over said hub and a proximal end of the needle.
